(19) United States
(12) Patent Application Publication
(10) Pub. No.: US 2004/0132676 A1
(43) Pub. Date: Jul. 8, 2004

(54) Polynucleotide formulation for enhanced intracellular transfer

(76) Inventor: Bruno Pitard, Reze (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/467,714

(22) PCT Filed: Feb. 19, 2002
(86) PCT No.: PCT/EP02/02617

(30) Foreign Application Priority Data
Feb. 19, 2001 (EP) 01420041.4

(51) Int. Cl. A61K 48/00; A61K 31/08

(52) U.S. Cl. 514/44; 514/723

(57) ABSTRACT

The invention relates to a pharmaceutical composition comprising a polynucleotide and at least 2% (weight/volume), preferably 2 to 10%, of a nonionic copolymer of formula (I)OH(CH2CH2O)a(CH(CH3)CH2O)a(CH2CH2O)cH, in which a, b, and c are such that the polyoxypropylene portion has a molecular weight of between 1450 and 2050, and the polyoxyethylene portions constitute between 75 and 85% (weight:weight) of the copolymer. The composition is preferably free of cationic lipid or of sodium phosphate. The copolymer is intended to improve the transfer of the polynucleotide into, or the expression of the polynucleotide in, eukaryotic cells. A typical example of a copolymer corresponding to formula (I) is F68. A composition according to the invention is in particular useful in the gene therapy, vaccination and immunotherapy fields.

The invention relates to a method for transferring a polynucleotide into eukaryotic cells and the use thereof in various therapeutic fields, including immunization.

Until the end of the 1980s, it was generally accepted that in vivo transfer of genetic information could be detected only if the genetic material was either encapsulated in liposomes or integrated into a viral vector. Then, as detection means improved, it was shown that expression of “naked DNA” may actually occur. This was demonstrated using DNA in isotonic aqueous solution, and injected intramuscularly or intradermally using a mere syringe (Wolff et al., Science (1990) 247: 1465). However, it became also apparent very soon that this discovery bears its own limitation in that high amounts of DNA were required. This was indeed a serious drawback that could impede development of what was called “the DNA technology” in the field of e.g. gene therapy or immunization. It was then proposed to move back and try to formulate the DNA with various chemical agents with the aim of increasing the efficiency of transfection into the cells and/or into the nucleus.

In the last ten years, an entire range of products have been tested to this effect. The most well-known compounds include PLGA microparticles, cationic lipids such as DOTMA (N-[1-2,2-(dioleyloxy)propyl]-N,N,N-trimethylammonium chloride), lipopolyamines and cationic polymers of the polylysine type. These compounds are able to bind polynucleotides and are supposed to promote the transfection thereof. However, none of them has given totally satisfactory results in the long term. Indeed, the effectiveness of the formulated polynucleotides was globally at most equivalent to that of nonformulated polynucleotides.

Some of these complexes may also unfortunately lack stability. Therefore it has been proposed to add a second compound, such as a nonionic surfactant, which stabilizes cationic transfecting agent/polynucleotide complexes (WO 98/34648). This surfactant may in particular be a polyoxyalkylene, such as Pluronics™ F68 marketed by BASF, also known under the name of Lutrol.

It has now been discovered that a compound such as F68 can also directly act on the level of transfection, in the absence of any cationic molecule, provided that it is added in a sufficient amount slightly higher than that required for stabilization purpose.

For this reason, the invention relates to a pharmaceutical composition comprising a polynucleotide and at least 2% (weight/volume) of a non-ionic copolymer of formula (I) OH(CH2CH2O)a(CH(CH3)CH2O)b(CH2CH2O)cH in which a, b and c are such that the polyoxypropylene portion has a molecular weight of from 1450 to 2050, and the polyoxyethylene portions constitute between 75 and 85% (weight:weight) of the copolymer.

In another embodiment, the invention also relates to the use of a nonionic copolymer of formula (I) OH(CH2CH2O)a(CH(CH3)CH2O)b(CH2CH2O)cH in which a, b and c are such that the polyoxypropylene portion has a molecular weight of between 1450 and 2050, and the polyoxyethylene portions constitute between 75 and 85% (weight:weight) of the copolymer, for the manufacture of a medicament containing a polynucleotide as active therapeutic agent; the copolymer being added to the polynucleotide at the concentration of at least 2% (weight volume) in order to improve the transfer of the polynucleotide into, and/or the expression of the polynucleotide in the cells of the individual in need of the medicament.

The invention also relates to a method for transferring a polynucleotide into eukaryotic cells, which comprises contacting cells with a polynucleotide formulated with at least 2% (weight volume) of a nonionic copolymer of formula (I) OH(CH2CH2O)a(CH(CH3)CH2O)b(CH2CH2O)cH, in which a, b and c are such that the polyoxypropylene portion has a molecular weight of from 1450 to 2050, and the polyoxyethylene portions constitute from 75 to 85% (weight:weight) of the copolymer.

Typically, it may be (i) a method for transferring a polynucleotide in vivo, which comprises administering a composition according to the invention to a mammal or any other animal; or (ii) a method for in vitro or ex vivo polynucleotide transfer, which comprises bringing into contact mammalian cells (or cells derived from any other animal) with a composition according to the invention. For ex vivo transfer method, mammalian cells are removed from an organism beforehand, and are further able to be reimplanted therein once they have incorporated the polynucleotide.

Advantageously, a composition according to the invention is free of cationic lipid and of sodium phosphate. Optionally, a composition according to the invention may also comprise a compound selected from the group consisting of sodium chloride, potassium chloride and magnesium chloride, preferably in an isotonic or hypertonic amount.

For use in present invention, a nonionic copolymer corresponding to the description given above is typically Pluronic™ F68 marketed by BASF. Its mean molecular weight is estimated at 8400 and the hydrophilic polyoxyethylene components represent approximately 80% of the total weight. This product is in solid form at 20° C. In aqueous solution at room temperature, a copolymer of formula (I) forms spherical micells within quite a wide concentration range (for example between 1 and 15%). At room temperature, an aqueous solution containing for example 1 to 15% of a copolymer of formula (I) is in the form of a liquid. At higher temperatures, the liquid solution turns into a gel and then a paste. More detailed information may be found in Alexandridis P., Current Opinion in Colloid & Interface Science (1997) 2: 478, which is incorporated by reference.

Advantageously, a pharmaceutical composition according to the invention comprises from 2 to 15%, preferably from 2 to 10%, most preferably about 5%, of a copolymer of formula (I).

For use in the present invention, the polynucleotide may be either a polydeoxyribonucleotide or a polyribonucleotide. Their origin does not matter: natural or artificial; genomic or complementary DNA; transfer or ribosomal RNA. They may in particular be of animal, human, plant, bacterial or viral origin.

Their function as a therapeutic agent may, in particular, consist in acting as an antisense molecule by controlling gene expression or mRNA transcription in the host cell. It may also be a polynucleotide capable of expressing a polypeptide e.g., a protein, of interest in an eukaryotic cell.

According to a particular embodiment, the polynucleotide operatively encodes a protein, polypeptide or peptide of therapeutic interest that upon expression by the host cell, is useful to overcome a dysfunction of the recipient organism. A composition according to the invention is therefore useful in in vivo or ex vivo gene therapy.

The polynucleotide may also operatively encode a polypeptide capable of generating an immune response against it in humans or animals; in particular the polypeptide is specific for a pathogenic organism (infectious agent) or a tumoral state (tumor-associated antigen).

According to this particular embodiment, the invention therefore leads to the preparation of vaccines or immunotherapeutic treatments applied to humans or to animals, in particular for treating or preventing infections, e.g. viral or bacterial infections, or cancers.

For use in these latter embodiments, the polynucleotide is advantageously DNA and is preferably in the form of a vector, i.a a plasmid vector. For safety reasons, such a vector is non-infectious and does not replicate in the host organism. Additionally, it substantially lacks the ability to integrate into the genome of the host organism. The DNA sequence encoding the therapeutic or antigenic polypeptide is placed under the control of elements required for its expression in the host organism. To this end, it is common practice to use the cytomegalovirus (CMV) early promoter.

As mentioned above, a pharmaceutical composition according to the invention may be used for the purposes of in vivo or ex vivo gene therapy. For this reason, according to another aspect, the invention also relates to a method for treating a disease induced by lack or deficiency of a gene, which comprises administering:

a composition comprising a polynucleotide which includes a gene able to correct the disease, and at least 2% (weight/volume) of a non-ionic copolymer of formula (I), to a patient in need such a treatment; or

collecting appropriate cells from a patient in need such a treatment, contacting these cells with a composition comprising (a) a polynucleotide encoding a gene able to correct the disease, and (b) at least 2% (weight/volume) of a nonionic copolymer of formula (I) so that the cells are transfected, and reimplanting the transfected cells into the patient.

A composition useful in gene therapy involves a polynucleotide comprising a therapeutic gene, i.e. a gene encoding a polypeptide that exhibits a therapeutic effect. This protein product may be homologous with respect to the target cell (i.e. a product which is normally expressed in the target cell when the latter exhibits no pathological condition). In this case, the expression of the polypeptide, subsequent to the administration of the composition according to the invention, makes it possible to overcome, for example, insufficient expression or the expression of an inactive or weakly active protein. The therapeutic gene may also encode a mutated form of a cellular poplypeptide with increased stability, modified activity, etc. The polypeptide may also be heterologous with respect to the target cell and, for example, may supplement, introduce or modify deficient or aberrant activity.

In view of this, the invention also relates to the combined use as described above of a polynucleotide encoding a polypeptide able to correct a gene deficiency and a non-ionic copolymer, in particular those of formula (I) or (II), in the manufacture of a medicament for treating a genetic disorder.

In in vivo therapeutic treatments, a composition according to the invention may be administered by the most suitable route for the treatment without any particular exclusion and, in general, it may be administered topically, cutaneously, orally, rectally, vaginally, parenterally, intranasally, intramuscularly, subcutaneously, intraocularly, intradermally, etc.

A composition according to the invention may also be useful in the field of immunization (e.g. vaccination) and immunotherapy. In this case, the composition involves a polynucleotide comprising a sequence encoding an antigenic product which is protein in nature (protein, polypeptide, peptide, etc.; globally referred to as polypeptide) and which may, for example, be a polypeptide expressed under natural conditions by any pathogenic organism or infectious agent (e.g. pathogenic virus or bacterium) or a mammalian polypeptide, the aberrant expression of which is characteristic of a tumoral state or condition (tumor-associated antigen). This is then referred to as an infectious agent-specific polypeptide or a cancer-specific polypeptide.

The use of a composition according to the invention for immunizing people is particularly advantageous since the non-ionic copolymer of formula (I) is able to significantly increase the antibody response against the antigen compared with that which is observed with a non-formulated polynucleotide. Indeed immunization using naked DNA or known DNA formulations generally raises an immune response of the cellular type, while the humoral response is poorly induced. Surprisingly, the composition of the invention have been found useful to induce a good antibody response.

Promoting the induction of an antibody response at a suitable level is not a specific property of the copolymer of formula (I). Non-ionic copolymers of various types (non-ionic polyols or derivatives) are also able to produce the same effect. To this end, the polyoxyalkylene within the polymer may in particular be polyoxyalkylene with alkylene groups of length or of conformation which may or may not be different; in particular, block copolymers of polyoxyethylene/polyoxypropylene, such as those described in Paschalis P. (above), e.g. poloxamers and poloxamines, and in particular those corresponding to the formula (II) OH(CH2CH2O)a(CH(CH3)CH2O)b(CH2CH2O)cH in which a b and c are such that the polyoxypropylene portion has a molecular weight between 1000 and 4000, and the polyoxyethylene portions constitute between 10 and 85% (weight:weight) of the copolymer.

In view of this, the invention also relates to the combined use as described above of a polynucleotide encoding a polypeptide specific for an infectious agent or for a cancer, and of a non-ionic copolymer, in particular those of formula (I) or (II), in the manufacture of a medicament for treating or preventing an infectious disease or a cancer. This pharmaceutical or immunization composition is in particular indicated for inducing an immune response, in particular of the humoral type, aimed at treating or preventing an infectious disease or a cancer.

A composition of the invention useful in the field of immunization and of immunotherapy may be administered by any route commonly used in these fields, in particular mucosally, e.g. orally, intragastrically and intranasally, or parenterally, e.g. intramuscularly, intradermally, intraepidermally and subcutaneously. Advantageously, a composition which is useful for treating a cancer is administered as close to the tumor site or tissue as possible. When the composition is aimed at treating a solid tumor, the administration may be carried out at the very site of the tumor, in particular by direct injection.

In general, the amount of polynucleotide to be administered depends on a large number of factors, such as the disease to be treated or prevented, the very nature of the polynucleotide, e.g. antisense RNA/DNA or plasmid DNA, the strength of the promoter of the plasmid vector, the biological activity of the product expressed by the gene, on the physical condition of the individual or of the animal, i.a of the mammal, for which the composition is intended (weight, age, etc.), on the method of administration and on the type of formulation. In general, a dose which is effective from a therapeutic or prophylactic point of view, of approximately 10 μg to approximately 5 mg, preferably of approximately 100 μg to approximately 5 mg, most particularly preferably from approximately 250 μg to approximately 3 mg, may be administered to adult humans. The administration may be carried out as a single dose or repeated at intervals.

A composition of the invention may be manufactured conventionally according to the regulations in use in the gene therapy, vaccines or immunotherapy field. In particular, a composition contains a pharma ceutically acceptable vehicle, and may be in solid e.g. lyophilized form, or liquid form. If necessary, the solid form may be reconstituted in liquid medium for administration.

The invention is illustrated hereinafter with reference to the following figures.

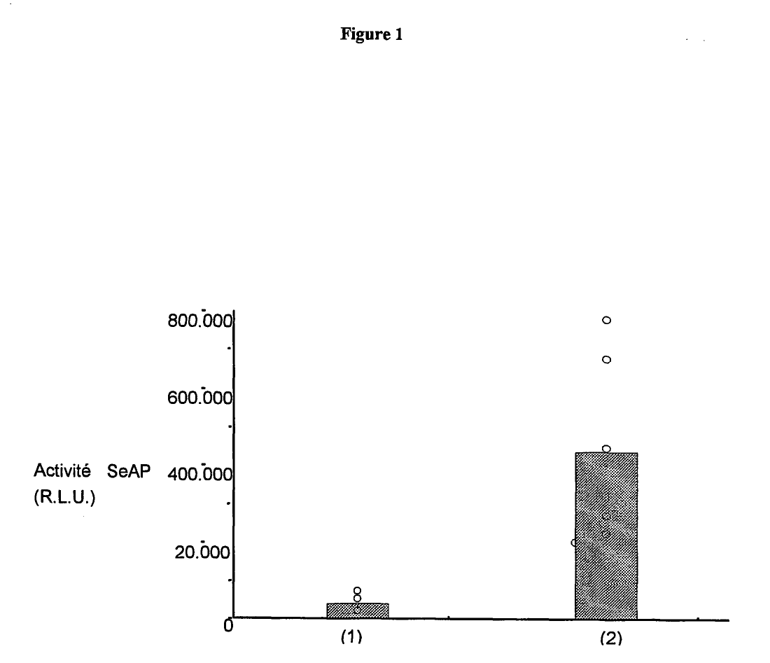

FIG. 1 shows the expression of secreted alkaline phosphatase (SeAP) generated in Balb/C mice by intramuscular injection of 10 μg of plasmid VR-SeAP, either naked (I) or formulated with 5% Lutrol (2). Two groups of 6 mice were constituted. 10 μg of plasmid under a volume of 10011 were administered to each mouse upon two concomitant injections (50 μl each) into each of the anterior Tibialis muscles. Seven days later, blood samples are recovered and the sera tested for SeAP activity.

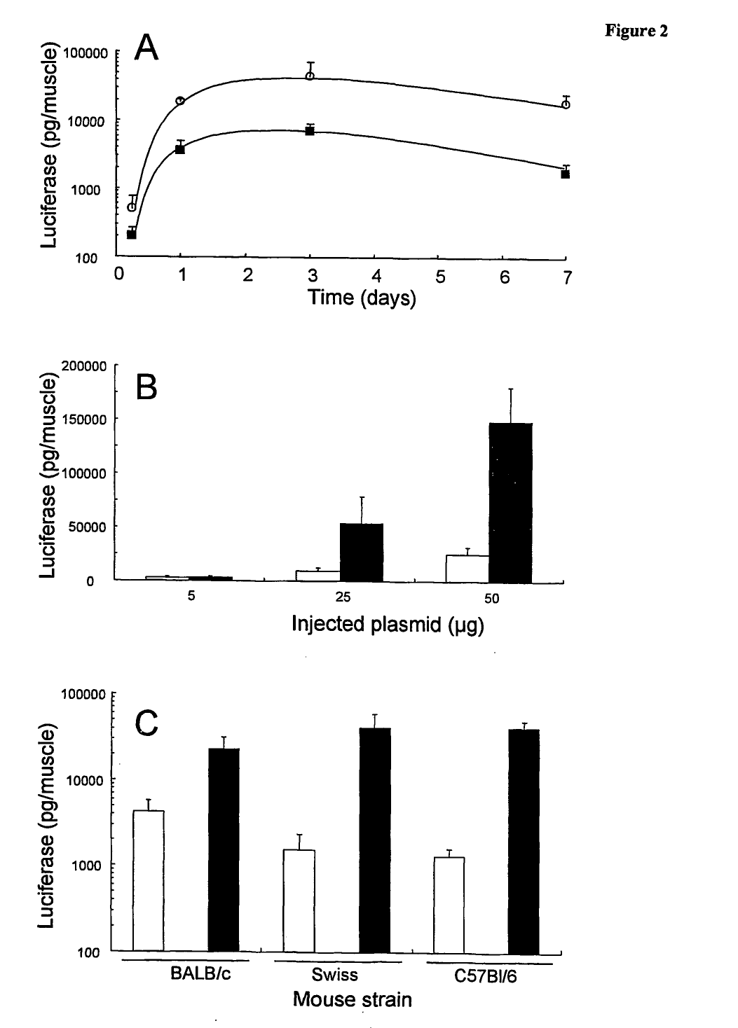

FIG. 2A shows the time-course of luciferase expression generated by intramuscular injection of plasmid pCMV-luc either naked (filled squares) or formulated in 5% Lutrol (open circles). Six tibial cranial muscles were included in each group. Each tibial cranial muscle of Swiss mice was injected with 5011 of saline solution containing 15 μg of DNA either naked (control group) or formulated. From 6 hours to 7 days later, the muscles were harvested and homogenized in the presence of lysis buffer to measure the luciferase activity. The results are expressed as the mean of the amount of luciferase synthetized in pg of luciferase per muscle. SEM: standard error of the mean.

FIG. 2B shows that luciferase expression is dependent on the amount of injected DNA. Two groups including 6 tibial cranial muscles were constituted. Each tibial cranial muscle of Swiss mice was injected with 50 μl of a preparation containing 5, 25 or 50 μg pCMV-luc, either naked (control group) or formulated with 5% Lutrol. The luciferase activity was measured 7 days later. The results are expressed as the of the amount of luciferase in pg per muscle.

FIG. 2C shows the influence of the mouse strain on the luciferase expression generated by intramuscular injection of 15 μg of plasmid pCMV-luc either naked (open bars) or formulated in 5% Lutrol (filled bars), under a volume of 50 μl. Groups of 6 tibial cranial muscles from various strains, i.e. Balb/C, Swiss and C57B1/6, were constituted. The luciferase activity was measured 7 days later. The results are expressed as the mean of luciferase produced in pg per muscle.

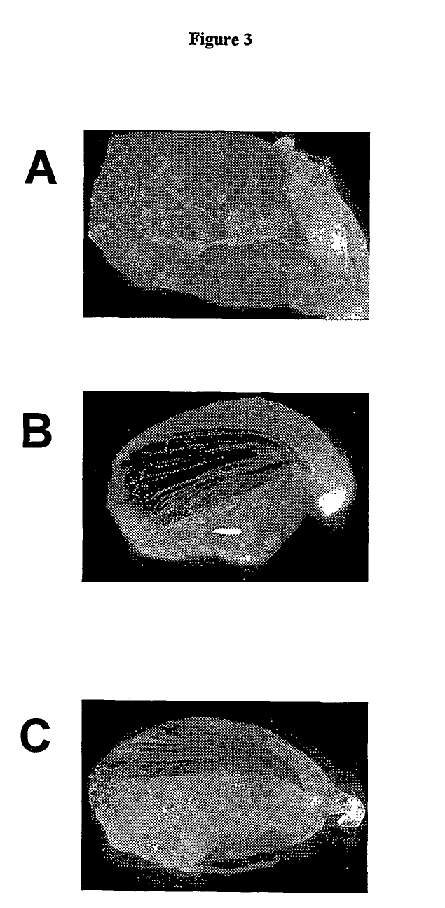

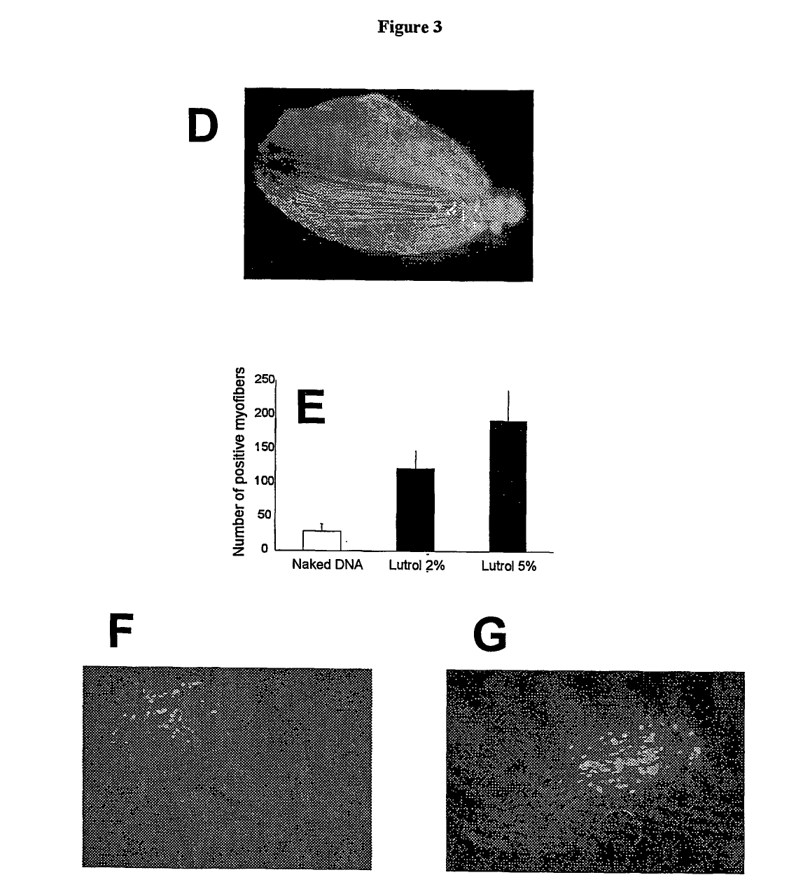

FIGS. 3A-D show the biodistribution of β-galactosidase expression, as visualized 7 days after the injection into Swiss mouse tibial cranial muscle of 50 μg of pCMVB-gal, either naked (3A) or formulated with 5% Lutrol (3B, C and D), under a volume of 50 μl.

FIG. 3E shows the average number of blue myofibers obtained following injection of 50 μg of pCMVβ-gal, either naked (open bar) or formulated with 2 or 5% Lutrol (black bars), under a volume of 50 μl. The tissue sections exhibiting the highest transfection level were used to count the number of blue cells. Data are presented as the mean+/−SEM with n=6 for each group.

FIGS. 3F and 3G depict the tissue section of tibial cranial muscles 7 days after injection of plasmid DNA pCMV-GFP either naked (3F) or formulated with 5% Lutrol (3G).

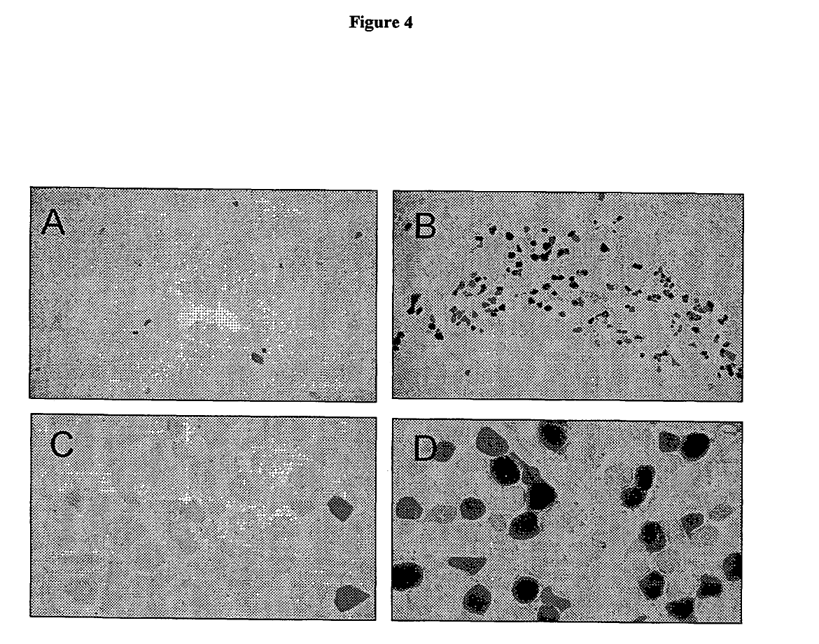

FIGS. 4A-D show the histological analysis of the β-galactosidase activity in rat tibial cranial muscles after intramuscular injection of 500 μg of pCMVB-gal either naked (4A and 4C) or formulated with 5% Lutrol (4B and 4D), under a volume of 500 μl. FIGS. 4C and 4D depict magnified views from 4A and 4B respectively.

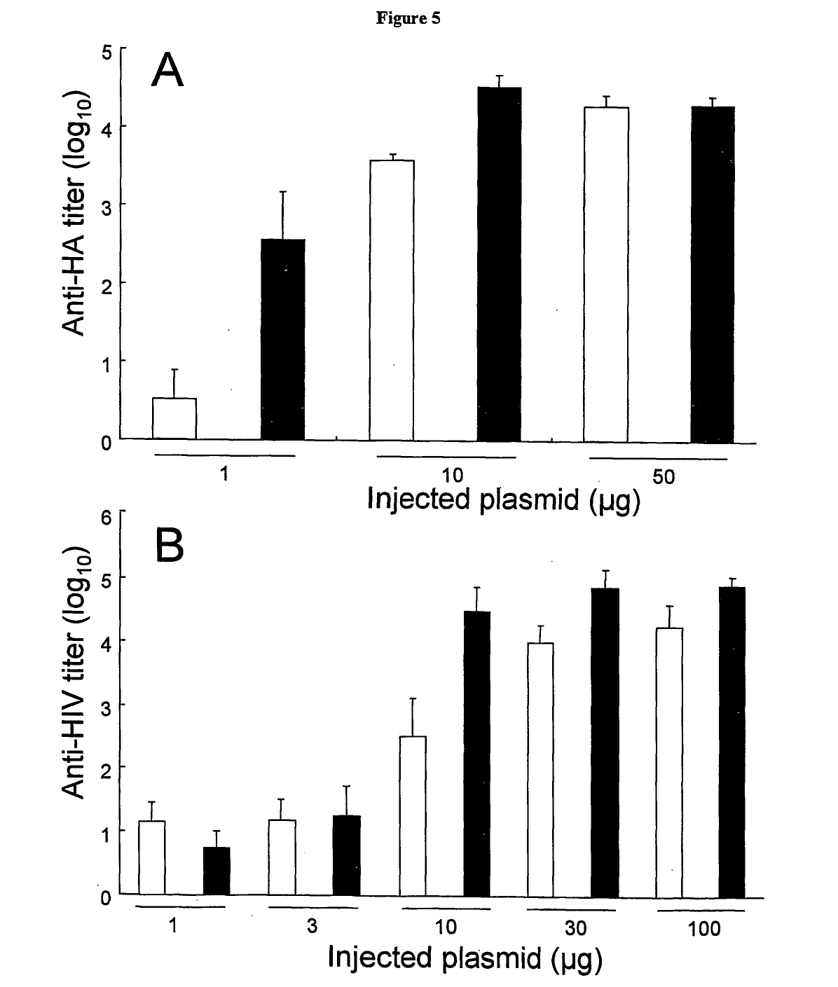

FIGS. 5A and 5B show the IgG antibody response to the A/PR/8/34 influenza strain (5A) or to HIV p24 (5B), respectively generated in Balb/C mice by various amounts of plasmids pCMV-HA (1, 10 and 50 μg, under a volume of 50 μl) and pM-1068 (1, 3, 10, 30 and 100 μg, under a volume of 50 μl), intramuscularly injected either naked (open bars) or formulated with 5% Lutrol (filled bars). Groups of 6 mice were constituted. Mice were administered a dose on days 0 and 21. Serum samples were recovered 2 weeks after the second injection and assayed by ELISA for anti-A/PR/8/34 (5A) and anti-p24 (5B) total IgG. Results were plotted as mean antibody titers, where error bars represent SEM.

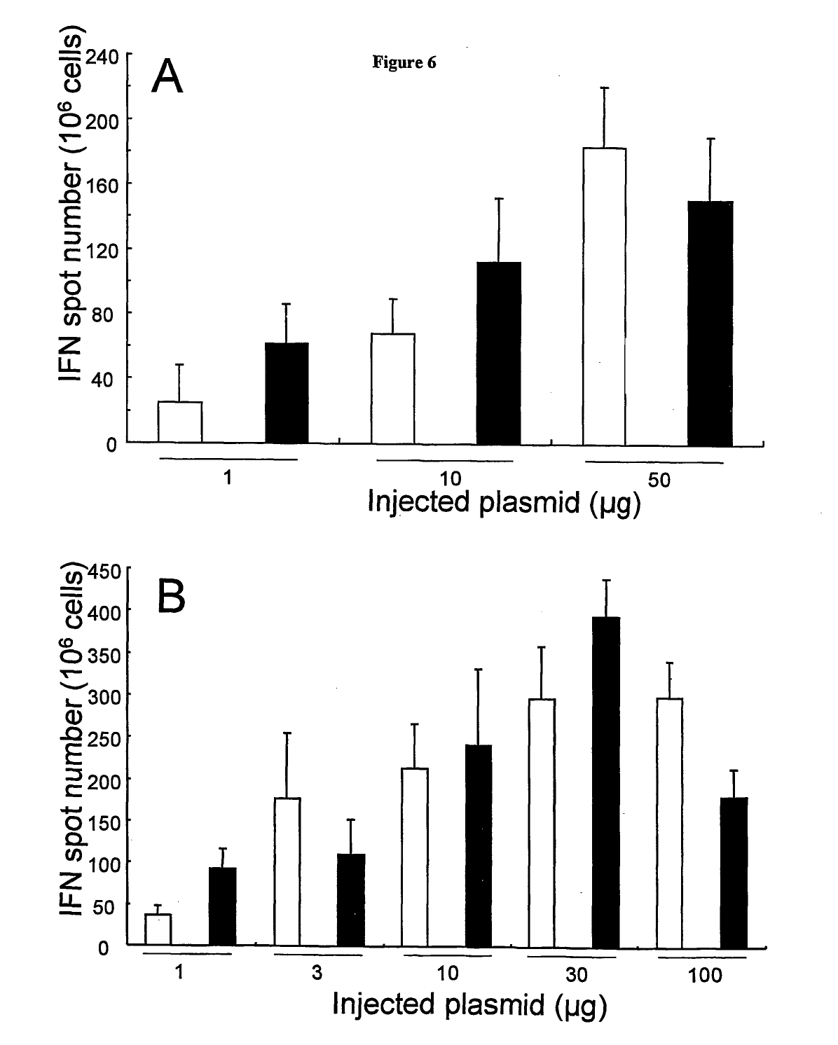

FIGS. 6A and 6B show the immune response of the γ-IFN producing cells to the A/PR/8/34 influenza strain (5A) or to H[V p24 (5B), respectively generated in Balb/C mice by various amounts of plasmids pCMV-HA (1, 10 and 50 μg, under a volume of 50 μl) and pM-1068 (1, 3, 10, 30 and 100 μg, under a volume of 50 μl), intramuscularly injected either naked (open bars) or formulated with 5% Lutrol (filled bars). Groups of 6 mice were constituted. Mice were administered a dose on days 0 and 21, and 12 days the second (booster) injection. Spleen cells were recovered and assayed by ELISPOT for γ-IFN producing cells. Each bar represents the average +/−SEM of the number of cells secreting γ-IFN per 10⁶ cells.

### EXPERIMENTS

### Materials and methods

Animals.

Female Balb/C mice were purchased from Charles River Laboratories (Les Oncins, France), Swiss, C57B1/6 and Wistar rats from Janvier Elevage (Le Genest St Isle, France). Mice and rats were used at 8-week-old and 400450 g respectively, housed and cared for in conformity with the guidelines of the French National Institutes of Health for animal experimentation.

Plasmids.

Plasmids pCMV-luc (Ferrari et al, Gene Ther. (1997) 4: 1100), pCMVB-gal (Clontech) pCMV-GFP (Clontech) and pM-1068 contain respectively the luciferase, the 13-galactosidase, the Green Fluorescent Protein and a synthetic gene encoding a HIV Gag/Pol/Nef fusion protein; each of them under the control of the human cytomegalovirus immediate early gene (CMV IE1) promoter. The UV chimera is constituted of a full-length codon-optimized gag gene (Deml et al, J. Virol. (2001) 75 (22): 10991) fused to peptides of the Pol and Nef proteins known to be T-cell epitopes immunodominant in humans. The same Pol and Nef epitopes are expressed by ALVAC-HIV vCP1456 (Jin et al, J. Virol. (2002) 76 (5): 2206).

Plasmids pCMV-SeAP (also referred to VR-SeAP) and pCMV-HA (VR-HA) were constructed using the VR1012 backbone originating from Vical (Hartikka et al, Hum. Gene Ther. (1996) 7 (10): 1205). They respectively contain the secreted alkaline phosphatase (SeAP) and the influenza virus hemagglutinin (HA) genes the under the control of the CMV IE1 promoter and the bovine growth hormone polyA signal.

Plasmids were purified from recombinant Escherichia coli by using EndoFree plasmid purification columns (Qiagen, Courtaboeuf, France). 0.8% agarose gel electrophoresis experiments indicated that plasmid was essentially supercoiled.

Formulation of Plasmid DNA with Lutrol.

Lutrol (also called F68), a poly(ethyleneoxide)₇₅-poly(propyleneoxide)₃₀-poly(ethyleneoxide)₇₅ block copolymer was a generous gift of BASF. Stock solutions were prepared at 20% (w/v) in water and stored at 4° C. Lutrol/plasmid DNA formulations were prepared by mixing equal volumes of Lutrol at 10% (2×F68) with 2×plasmid DNA solution in 300 mM NaCl (1.8% NaCl), 50 mM Hepes buffer, to reach the required final concentration of DNA per 50 μl solution containing 5% (w/v) Lutrol in 150 mM NaCl (0.9%). Naked DNA solution in 150 mM NaCl, are also used as control.

Intramuscular Injection.

Mice were anaesthetised by intraperitoneal injection of 400 μl Etomidate (Hypnomidate 2 mg/ml, Janssen-Cilag, Issy-les-Moulineaux, France) or a mixture of Ketamine (Imalgen 500, Merial, Lyon, France) and Xylazine (Rompun 2%, Bayer Puteaux, France). The skin overlying the tibial cranial muscle was shaved and the animals were injected with 5011 of naked or formulated DNA For DNA immunization, Balb/C mice were injected intramuscularly two times at 3-week interval. Rats were anaesthetised by intraperitoneal injection of Zoletil ND (Virbac, Carros, France) at 30 mg/kg. Five hundred μl containing 500 μg of DNA, naked or formulated, were injected into the rat tibial cranial muscle.

Measurement of Alkaline Phosphatase Activity.

The SeAP was assayed using the Clontech kit ref. Great Escape K-2041-1. Briefly, 151 μl of serum are distributed per well in a 96-well plate and 45 μl of 1× dilution buffer are added. Plate is incubated for 30 min at 65° C. in order to inactivate endogenous alkaline phosphatase and then, cooled to 4° C. and left to stand at room temperature. Sixty μl per well of test buffer are then added and plate is incubated for 5 min at room temperature. The content of the wells is transferred into a black 96-well plate (Microfluor, Dynatech). 60 μl of CSPD (disodium 3-(4-methoxy-spiro(1,2-dioxetane-3,2′-(5′-chloro)tricyclo 3,3.1.1^{3.7}]decan)-4-yl)phenyl phosphate) diluted 20-fold are then added, followed by incubation for 20 min at room temperature. The chemiluminescence signal, which is a reflection of the alkaline phosphatase activity, is measured using a Victor-1420 luminometer (Wallac).

A standard range (dilutions of 10⁻⁷ to 10⁻¹, i.e. from 0.01 to 10,000 pg/μl) of SeAP is included using the recombinant SeAP at 0.1 mg/ml as provided in the kit. The regression line for this standard range makes it possible to convert the SeAP expression into pg/ml, using light units (RLU).

Measurement of Luciferase Activity.

Unless otherwise stated, mice were killed 7 days after tibial cranial muscle injection of DNA. Each injected muscle was removed, frozen in liquid nitrogen and homogenized in 1 ml of Reporter Lysis Buffer (Promega, Charbonnières, France) supplemented with a protease inhibitor cocktail (Complete, Roche Diagnostics, Mannheim, Germany). After centrifugation at 1000 rpm for 4 min luciferase activity was measured on 10 μl supernatant with a Victor2 (Perkin Elmer, Les Ullis, France). Light emission was measured for a period of 5 sec. after the addition of 100 μl of luciferase substrate (Promega, Charbonnieres, France). A standard curve prepared in an uninjected mouse tibial cranial muscle was included on each microplate, using purified luciferase (Sigma). Results were expressed as pg luciferase per muscle +/−SEM.

Histological Analysis.

Seven days after intramuscular injection of pCMV-Bgal plasmid DNA/Lutrol complexes, tibial cranial muscles were fixed for 20 minutes in 4% fresh paraformaldehyde, washed three times for 30 minutes in PBS containing MgCl₂ 2 mM, sodium deoxycholate 0.01%, NP40 0.4% at pH 7.4. Muscles were then incubated overnight at 37° C. in X-gal solution (MgCl₂ 2 mM sodium deoxycholate 0.01%. NP40 0.4% potassium ferricyanide 5 mM, potassium ferrocyanide 5 mM in PBS pH 7.4) containing 1 mg/ml of 5-bromo-4-chloro-3-indolyl-β-D25 galactopyranoside (Euromedex, France). Tissues were finally paraffin embedded, and cut into 4 μm sections. One section every 200 μm was mounted and counterstained with Kernechtrot solution.

Measurement of Antibody Response.

The humoral response is studied using an ELISA assay as described in Haensler et al, Vaccine

17 (7-8): 628, in which the inactivated influenza virus A/PR/8/34 or recombinant HIV P24 (a Gag protein) is used as coating antigen.

Serum samples were collected from anaesthetized mice before immunization and 14 days after the second DNA formulation injection. Antibodies (total IgG and/or IgG subclasses) specific for influenza virus and for P24 were measured by ELISA. The whole-inactivated influenza virus (A/PR/8/34) and recombinant P24 were used to coat the wells. Serum samples were diluted from 1/100 to 1/204800. Peroxidase-conjugated goat anti-mouse IgG (Jackson ImmunoResearch Laboratories, Interchim, Montluçon, France) was diluted 1/30000. Plates were read using a spectrophotometer at 490-650 nm (Vmax plate reader, Molecular Devices, BioTime, St-Gregoire, France). Average of the blanks was subtracted from experimental data. Anti-influenza virus and anti-P24 titers were calculated from a 4-parameter regression curve of a standard A/PR/8/34 and P24-specific mouse serum included in each ELISA plate, respectively. Titer of the standard had been previously determined from 10 independent experiments according to the formula: OD(490-650 nm)×10/1/dilution. An antiserum was considered positive if its specific titer after immunization was at least 0.5 log₁₀ higher than the average of the preimmune titer.

ELISPOT.

Nitrocellulose-backed microtiter plates (96-well Multiscreen MA plate, Millipore St Quentin Fallavier, France) were coated with primary anti γ-IFN antibody (# 18181D Pharmingen, Pont de Claix, France) at 10 μg/ml in phosphate buffered saline (PBS) for 1 hr at room temperature. Plates were blocked with RPMI medium (Gibco BRL, Life Technologies, Cergy Pontoise, France) for 1 hr and washed with PBS. Splenocytes harvested 12 days after the second immunization were resuspended (at a concentration of 2.10⁶ cells/ml for splenocytes from HA DNA immunized mice or 4.10⁶ cells/ml for splenocytes from HIV DNA immunized mice) in RPMI media containing 10% foetal calf serum (FCS) and antibiotics. Mouse IL-2 (Boehringer) was added to a final concentration of 20 U/ml. One hundred μl of the cell suspensions were distributed in triplicate in anti-cytokine-coated plates. Peptides corresponding to the A/PR/8/34 HA2 cytotoxic T lymphocytes (CTL) epitope restricted by the H-2 Kd molecule (IYSTVASSLVL) or the A/PR/8/34 nucleoprotein CTL epitope (TYQRTRALVTG, negative control) were added to splenocytes from HA DNA immunized mice at a final concentration of 20 μg/ml in a final volume of 200 μl. Peptides corresponding to the gag cytotoxic T lymphocytes (CTL) epitope restricted by the H-2 Kd molecule (AMQMLKETI) or the A/PR/8/34 nucleoprotein CTL epitope (TYQRTRALVTG, negative control) were added to splenocytes from HIV DNA immunized mice at a final concentration of 20 μg/ml in a final volume of 200 μl. Plates were incubated for 18 hr at 37° C. in a humidified 5% CO₂ incubator. Plates were then washed with PBS containing 0.05% Tween 20 and coated with a biotinylated anti γ-IFN antibody (#18112D Pharmingen, France) at 1 μg/ml for 2 hrs at room temperature. Plates were then washed with PBS Tween and treated with a peroxydaseconjugated streptavidin (#7100-05 Southern Biotechnology, Clinisciences, Montrouge, France). Plates were incubated for 1 hr at room temperature and thoroughly washed. After a final wash, γ-IFN secreting cells were visualized by the addition of a solution of 3-amino-9-ethylcarbazole peroxide substrate. Spots were counted with an image analyzer (Microvision Instruments, Evry, France) and values were confirmed by manual counting. The frequency of γ-IFN producing cells was calculated by averaging the number of spots for triplicate wells. Means of spots from no-antigen wells (less than 10 γ-IFN spots) were substracted.

### Results

Transfection Activity of Lutrol/DNA Formulations

The level of expression of the gene encoding the SeAP or the luciferase was studied after in vivo transfection in the presence or absence of 5% F68.

As shown in FIG. 1, the SeAP activity measured in serum samples indicates that the presence of 5% F68 improves the level of expression of SeAP about 1′-fold.

As shown in FIG. 2, the luciferase activity measured in transfected mouse tibial cranial muscle indicates that Lutrol/DNA formulations are more active in promoting luciferase expression than naked DNA. The time-course of luciferase expression shows (FIG. 2A) that luciferase was already synthetized 6 hours after intramuscular injection of DNA or Lutrol/DNA formulations. Luciferase expression increases progressively and reaches, at day 3, a plateau. At day 7, a slight decrease in luciferase activity is detected. At these 4 time points, the luciferase activity is greater with Lutrol/DNA formulations that with naked DNA (from 2.5 fold at 6 hours to 11 fold at day 7). Luciferase expression increases linearly as a function of the amount of plasmid DNA injected into the mouse tibial cranial muscle, for instance 50 μg of naked DNA results in 9 times greater luciferase activity than the activity of 5 μg naked DNA (FIG. 2B). On the opposite, Lutrol/DNA formulations injected into tibial cranial muscle lead to luciferase expression that is not directly proportional to the injected amount of plasmid, because 50 μg of DNA formulated with 5% Lutrol lead to 60 times greater luciferase expression than 5 μg of formulated DNA. The study of luciferase expression with naked DNA or formulated with 5% Lutrol injected into mouse tibial cranial muscles of Balb/C, Swiss and C57B1/6 shows (FIG. 2C) that gene expression is improved by the presence of 5% Lutrol, irrespective of the mouse strain.

In Vivo Biodistribution of Reporter Gene Expression

Histological analysis of mouse tibial cranial muscles 7 days after intramuscular injection of naked DNA (FIG. 3A) or Lutrol/DNA formulation (FIGS. 3B, C and D) with β-galactosidase reporter gene shows that Lutrol/DNA associations increase the number of muscle fibers that express the β-galactosidase enzyme. The number of β-galactosidase-expressing myofibers was counted for each mouse tissue sections injected with naked DNA or with DNA formulated with 2 and 5% Lutrol. The number of positive fibers is 4 and 10 fold higher with DNA formulated with respectively 2 and 5% Lutrol than those obtained with intramuscular injection of naked DNA (FIG. 3F). Examination of tissue sections shows that the level of transgene expression is variable from a myofiber to another, and that on average, myofibers transfected with Lutrol/DNA formulation express the β-galactosidase at a higher level than those transfected with naked DNA. Analysis of the β-galactosidase activity in muscles close to the tibial cranial shows that transgene expression is restricted to the injected muscle. To strengthen the observation that Lutrol enhances the number of transgene expressing myofibers, this protocol was repeated using Green Fluorescent Protein instead of β-galactosidase. Again, tissue sections shows that 5% Lutrol mixed with plasmid DNA leads to an increase of the number of myofibers that express the GFP (FIGS. 3F and G).

In order to validate our gene delivery system on an other animal species, tibial cranial muscles of rat were injected with naked pCMVB-gal plasmid DNA or Lutrol/DNA formulations.

Microscopic examination of tibial cranial muscles from rats injected with Lutrol/DNA formulations (FIG. 4B) shows a significant increase in the percentage of transfected fibers compared with the muscle which received naked DNA (FIG. 4A). Variable blue intensity is observed in light micrographs of representative rat tibial cranial muscle sections, indicating that the level of β-galactosidase expression is different from a myofiber to another. Higher magnification of these tissue sections shows that β-galactosidase enzyme was also found in the vicinity of the cells in the endomysium and in the capillaries (FIG. 4D).

DNA Immunization in the Presence of Lutrol

To evaluate the influence of Lutrol on DNA immunogenicity, a plasmid encoding the HA protein of the influenza virus A/PR/8/34 (H1N1) under the control of the CMV promoter was injected into the mouse tibial cranial muscle and serum titers were measured by ELISA 5 weeks later (FIG. 5A).

Injection of 1 μg of naked HA plasmid results in modest specific IgG antibodies titer, and seroconversion is not observed in the 6 mice tested. By contrast, the magnitude of the anti-HA titer is increased 110-fold by intramuscular injection of Lutrol/DNA formulations containing 1 μg DNA with seroconversion in 4 of 6 animals. For the 10 μg dose, Lutrol/DNA formulation increases the mean geometric titer of positive antisera by 9 fold (from 4.52 to 3.58 log₁₀) and all the mice seroconvert. Immunization with 50 μg of plasmid DNA formulated with Lutro does not improve either the antibody titer or the number of serconverting mice. Previous experiments have showed that intramuscular injection of 50 μg plasmid DNA results in a plateau level of specific anti IGg titer and that the percentage of seroconversion is 100% (data not shown).

Another model of plasmid DNA immunization was used to confirm that determine if Lutrol/DNA formulation can actually enhance an immune response. A plasmid DNA encoding HIV gag, po1 and nef proteins was used (pM-1068). Balb/C mice were immunized by intramuscular injections of various plasmid DNA concentrations in saline or formulated with 5% Lutrol, and sera were collected after 5 weeks for antibody anti-p24 titer measurement. FIG. 5B shows that specific IgG antibodies are very low in mice injected with 1 and 3 μg of plasmid DNA either in saline or formulated with 5% Lutrol. Higher responses are obtained with 10, 30 and 100 μg of naked plasmid DNA, with seroconversion in 3 of 6, 6 of 6 and 6 of 6 mice, respectively. Intramuscular injection of Lutrol/DNA formulations containing 10, 30 and 100 μg of plasmid DNA leads respectively to an increase of the mean antibody titer by a factor of 100, 10 and 4 over naked DNA. For the 10 μg dose, the presence of 5% Lutrol increases the number of seroconverting animal from 3 of 6 to 6 of 6.

Altogether these results with two immunization models confirm that Lutrol is useful to significantly enhance the humoral response induced by DNA immunization.

Cellular Responses After Intramuscular Injection of Lutrol/DNA Formulations

The effect of Lutrol on the cellular response induced by DNA immunization was studied 2 and 3 weeks after the booster injection of plasmids pCMV-HA and pM-1068, respectively. Spleen cells were harvested and tested by ELISPOT for the presence of HA or p24-specific T-cells producing γ-IFN upon stimulation with a peptide containing a CTL epitope. FIGS. 6A and B showed a clear dependence of the amount of DNA injected into the tibial cranial on the number of spots. The mean response are higher in the presence of 5% Lutrol, but the differences are not statistically significant.

1. A pharmaceutical composition which comprises a polynucleotide and at least 2% (weight/volume) of a non-ionic copolymer of formula (I) OH(CH2CH2O)a(CH(CH3)CH2O)b(CH2CH2O)cH in which a, b and c are such that the molecular weight of the polyoxypropylene portion is from 1450 and 2050, and the polyoxyethylene portions constitute from 75 to 85% (weight:weight) of the copolymer.

2. The composition according to claim 1, which is free of cationic lipid.

3. The composition according to claim 1 or 2, which is free of sodium phosphate.

4. The composition according to claim 3, which comprises a polynucleotide and 2 to 10% (weight/volume) of a non-ionic copolymer of formula (I).

5. The composition according to any one of claims 1 to 4, which comprises a polynucleotide and about 5% (weight/volume) of a non-ionic copolymer of formula (I).

6. The composition according to any one of claims 1 to 5, which further comprises a compound selected from the group consisting of sodium chloride, potassium chloride and magnesium chloride, in an isotonic or hypertonic amount.

7. The composition according to any one of claims 1 to 6, in which the non-ionic copolymer is Pluronics™ F68.

8. The composition according to any one of claims 1 to 7, in which the polynucleotide is an antisense polynucleotide.

9. The composition according to any one of claims 1 to 7, in which the polynucleotide is capable of expressing a polypeptide of interest, in a eukaryotic cell.

10. The composition according to claim 9, in which the polynucleotide is able to express a polypeptide specific for a pathogenic organism or a tumoral state.

11. The composition according to claim 9, in which the polynucleotide is able to correct a gene deficiency.

12. The composition according to any one of claims 8 to 11, in which the polynucleotide is DNA.

13. The use of a non-ionic copolymer of formula (I) OH(CH2CH2O)a(CH(CH3)CH2O)b(CH2CH2O)cH, in which a, b and c are such that the polyoxypropylene portion has a molecular weight of between 1450 and 2050, and the polyoxyethylene portions constitute between 75 and 85% (weight:weight) of the copolymer, in the manufacture of a medicament containing a polynucleotide as active therapeutic agent; the copolymer being added to the polynucleotide at the concentration of at least 2% (weight:volume), in order to improve the transfer of the polynucleotide into, and/or the expression of the polynucleotide in the cells of the patient in need of such medicament.

14. The use according to claim 13, in which the copolymer is added to the nucleotide at the concentration of from 2 to 10%.

15. The use according to claim 14, in which the copolymer is added to the nucleotide at the concentration of about 5%.

16. The use according to any one of claims 13 to 15, in which the medicament is free of cationic lipid or of sodium phosphate.

17. The use according to any one of claims 13 to 16, in which the medicament further comprises a compound selected from the group consisting of sodium chloride, potassium chloride and magnesium chloride, in an isotonic or hypertonic amount.

18. The use according to any one of claims 13 to 17, in which the copolymer is Pluronics™ F68.

19. The use according to any one of claims 13 to 18, in which the polynucleotide is defined as in any one of claims 8 to 12.

20. The use of a non-ionic copolymer of formula (I) OH(CH2CH2O)a(CH(CH3)CH2O)b(CH2CH2O)cH, in which a, b and c are such that the polyoxypropylene portion has a molecular weight of between 1450 and 2050, and the polyoxyethylene portions constitute between 75 and 85% (weight:weight) of the copolymer, in the manufacture of a medicament containing a polynucleotide as active therapeutic agent which is able to express in an eukaryotic cell, a polypeptide specific for a pathogenic organism or a tumoral state, for inducing an immune response against the pathogenic organism or tumoral state; the copolymer being added to the polynucleotide at a concentration of at least 2% (weight: volume).

21. The use according to claim 20, in which the immune response is, in particular, of the humoral type.

22. The use according to claim 20 or 21, in which the immune response protects against a tumoral state or an infection induced by the pathogenic organism.

23. The use according to claim 20 or 21, in which the immune response has a therapeutic action against a tumoral state or an infection induced by the pathogenic organism.

24. The use of a non-ionic copolymer of formula (I) OH(CH2CH2O)a(CH(CH3)CH2O)b(CH2CH2O)cH, in which a, b and c are such that the polyoxypropylene portion has a molecular weight of between 1450 and 2050, and the polyoxyethylene portions constitute between 75 and 85% (weight:weight) of the copolymer, in the manufacture of a medicament containing a polynucleotide as active therapeutic agent which operatively encodes in eukaryotic cells, a polypeptide able to correct a gene deficiency, for treating a genetic disorder; the copolymer being added to the polynucleotide at a concentration of at least 2% (weight:volume).

25. A method for transferring a polynucleotide into eukaryotic cells, which comprises contacting the cells with the polynucleotide, the polynucleotide being formulated with at least 2% (weight:volume) of a non-ionic copolymer of formula (I) OH(CH2CH2O)a(CH(CH3)CH2O)b(CH2CH2O)cH, in which a, b and c are such that the polyoxypropylene portion has a molecular weight of from 1450 to 2050, and the polyoxyethylene portions constitute from 75 to 85% (weight:weight) of the copolymer.

26. The method according to claim 25, in which the polynucleotide is formulated as described in any one of claims 2 to 6.

27. The method according to claim 25 or 26, in which the copolymer is Pluronics™ F68.

28. The method according to claim 25, 26 or 27, in which the polynucleotide is defined as in any one of claims 8 to 12.
